(19) United States
(12) Patent Application Publication
(10) Pub. No.: US 2026/0090740 A1
(43) Pub. Date: Apr. 2, 2026

(54) WEARABLE DEVICE WITH BLOOD OXYGEN MEASUREMENT FUNCTION AND BLOOD OXYGEN MEASUREMENT METHOD

(71) Applicant: PixArt Imaging Inc., Hsin-Chu City (TW)

(72) Inventors: Chih-Hao Wang, Hsin-Chu City (TW); Shih-Jen Lu, Hsin-Chu City (TW); Yang-Ming Chou, Hsin-Chu City (TW); Chien-Yi Kao, Hsin-Chu City (TW); Hung-Chih Wang, Hsin-Chu City (TW); Hsin-Yi Lin, Hsin-Chu City (TW)

(73) Assignee: PixArt Imaging Inc., Hsin-Chu City (TW)

(21) Appl. No.: 18/898,694

(22) Filed: Sep. 27, 2024

(51) Int. Cl. A61B 5/1455; A61B 5/00

(52) U.S. Cl. CPC A61B 5/14551; A61B 5/6802

(57) ABSTRACT

A wearable device with a blood oxygen measurement function, comprising: at least one light source, configured to emit light; an optical sensor, configured to sense an optical signal generated according to reflected light of the light; a pressure sensor, configured to sense a pressure provided by a user wearing the wearable device; a processing circuit, configured to calibrate the optical signal to generate a calibrated optical signal, and configured to compute a blood oxygen level according to the calibrated optical signal; and a pressure adjusting structure, configured to adjust an internal wearing space of the wearable device, wherein the pressure changes corresponding to the internal wearing space. A blood oxygen measurement method which the wearable device can use is also disclosed. Thereby the optical signal may be calibrated corresponding to the state of the wearable device. By this way, the blood oxygen measurement may be more accurate.

### BACKGROUND OF THE INVENTION

### 1. Field of the Invention

The present invention relates to a wearable device with a blood oxygen measurement function and a blood oxygen measurement method, and particularly relates to a wearable device with a blood oxygen measurement function and a blood oxygen measurement method which can calibrate the blood oxygen measurement according to the state of the wearable device.

### 2. Description of the Prior Art

With the advancement of technology, some wearable devices have the function of measuring blood oxygen. This type of wearable device can use optical signals to measure blood oxygen. However, users have different usage habits when wearing wearable devices, which may affect the accuracy of blood oxygen measurement. For example, some users will adjust a watch to be tight when wearing the watch, while some users will adjust the watch to be loose when wearing the watch. This may affect the optical signal and thus affects the accuracy of blood oxygen measurement.

### SUMMARY OF THE INVENTION

One objective of the present invention is to provide a wearable device which can calibrate the blood oxygen measurement corresponding to the state of the wearable device.

Another objective of the present invention is to provide a blood oxygen measurement method which can calibrate the blood oxygen measurement corresponding to the state of the wearable device.

One embodiment of the present invention provides a wearable device with a blood oxygen measurement function, comprising: at least one light source, configured to emit light; an optical sensor, configured to sense an optical signal generated according to reflected light of the light; a pressure sensor, configured to sense a pressure provided by a user wearing the wearable device; a processing circuit, configured to calibrate the optical signal to generate a calibrated optical signal, and configured to compute a blood oxygen level according to the calibrated optical signal; and a pressure adjusting structure, configured to adjust an internal wearing space of the wearable device, wherein the pressure changes corresponding to the internal wearing space.

Another embodiment of the present invention provides a blood oxygen measurement method, applied to a wearable device with at least one light source, an optical sensor and a pressure sensor, comprising: (a) emitting light by the light source; (b) sensing an optical signal generated according to reflected light of the light by the optical sensor; (c) sensing a pressure provided by a user wearing the wearable device by the pressure sensor; (d) calibrating the optical signal according to the pressure to generate a calibrated optical signal; and (e) computing a blood oxygen level according to the calibrated optical signal; wherein the wearable device further comprises a pressure adjusting structure configured to adjust an internal wearing space of the wearable device, wherein the pressure changes corresponding to the internal wearing space.

In view of above-mentioned embodiments, the optical signal may be calibrated corresponding to the state of the wearable device. By this way, the blood oxygen measurement may be more accurate.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 and FIG. 2 are schematic diagrams illustrating different states of the wearable device, according to embodiments of the present invention.

FIG. 3A is a schematic diagram illustrating a wearable device according to one embodiment of the present invention.

FIG. 3B is a schematic diagram illustrating a wearable device according to another embodiment of the present invention.

FIG. 4 and FIG. 5 are schematic diagrams illustrating the first signal component, the second signal component and the third signal component in different states of the wearable device, according to embodiments of the present invention.

FIG. 6 is a flow chart illustrating a calibration method for the first signal component or the second signal component, according to embodiments of the present invention.

FIG. 7 is a flow chart illustrating a blood oxygen measurement method according to one embodiment of the present invention.

### DETAILED DESCRIPTION

In the following descriptions, several embodiments are provided to explain the concept of the present application. The term “first”, “second”, “third” in following descriptions are only for the purpose of distinguishing different one elements, and do not mean the sequence of the elements. For example, a first device and a second device only mean these devices can have the same structure but are different devices.

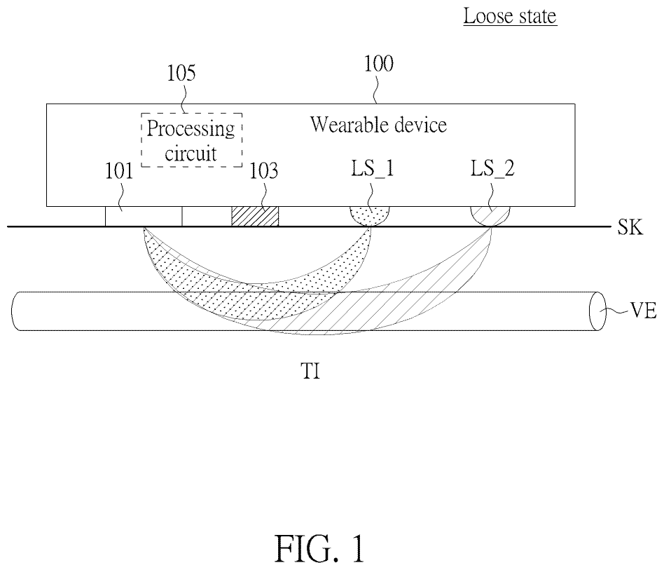

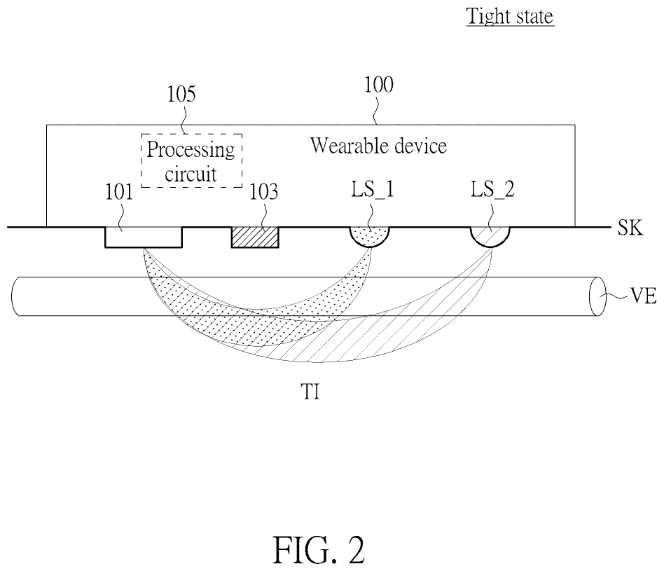

FIG. 1 and FIG. 2 are schematic diagrams illustrating different states of the wearable device, according to embodiments of the present invention. As shown in FIG. 1, the wearable device 100 comprises an optical sensor 101, a pressure sensor 103, a processing circuit 105 and at least one light source. The light source is configured to emit light. In the embodiments of FIG. 1 and FIG. 2, a first light source LS_1 and a second light source LS_2 are provided. Also, in one embodiment, the light emitted from the first light source LS_1 and the second light source LS_2 have different light wave lengths. For example, the first light source LS_1 emits red light and the second light source LS_2 emit infrared light. However, the type and the number of the light source are not limited to the embodiments of FIG. 1 and FIG. 2.

The optical sensor 101 is configured to sense an optical signal generated according to reflected light of the light. The pressure sensor 103 is configured to sense a pressure provided by a user wearing the wearable device 100. The processing circuit 105 is configured to compute a calibrated blood oxygen level according to the pressure. For example the processing circuit 105 is configured to calibrate the optical signal to generate a calibrated optical signal according to the pressure, and configured to compute a blood oxygen level according to the calibrated optical signal. The wearable device 100 further comprises a pressure adjusting structure configured to adjust an internal wearing space of the wearable device, wherein the pressure changes corresponding to the internal wearing space. More specifically, the smaller the internal wearing space, the larger the pressure is. Oppositely, the larger the internal wearing space, the smaller the pressure is. Details of the optical signal and the pressure adjusting structure will be described in following embodiments.

The wearable device 100 is in different states in FIG. 1 and FIG. 2. In FIG. 1, the wearable device 100 is in a loose state, which means the above-mentioned internal wearing space is larger and the user provides a smaller pressure to the wearable device 100. On the contrary, in FIG. 2, the wearable device 100 is in a tight state, which means the above-mentioned internal wearing space is smaller and the user provides a larger pressure to the wearable device 100.

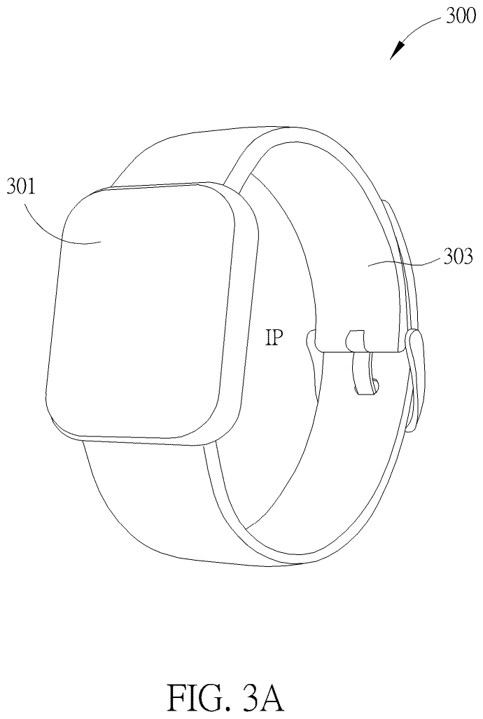

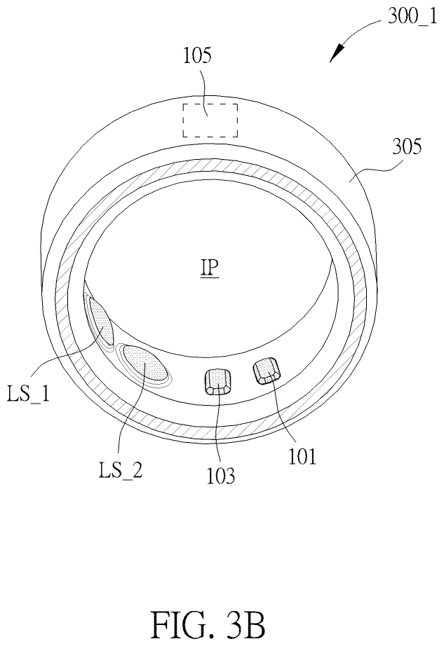

FIG. 3A is a schematic diagram illustrating a wearable device according to one embodiment of the present invention. In the embodiment of FIG. 3A, the wearable device 100 is a smart watch 300 which comprises a watch case 301 and a watch strap 303. The optical sensor 101, the pressure sensor 103, the processing circuit 105, the first light source LS_1 and the second light source LS_2 may be provided inside or on the watch case 301. Also, the watch strap 303 is the above-mentioned pressure adjusting structure, which can be used to adjust the internal wearing space IP, which affects the pressure that the pressure sensor 103 senses. Please note, the wearable device 100 used in the present invention is not limited to the smart watch 300 illustrated in FIG. 3A. Any wearable device 100 which can perform the same function falls in the scope of the present invention. For example, the wearable device 100 can be a wristband and the pressure adjusting structure may be a ring of the wristband or an inflatable cuff attached to the ring.

FIG. 3B is a schematic diagram illustrating a wearable device according to another embodiment of the present invention. In the embodiment of FIG. 3B, the wearable device is a ring 300_1. As shown in FIG. 3B, the wearable device 300_1 may comprise a first adjustable structure, an enclosed outer ring 305, the first light source LS_1, the second light source LS_2, the optical sensor 101, the pressure sensor 103 and the processing circuit 105. In one embodiment, the enclosed outer ring 305 may be made of solid, such as wood, plastic, ceramics or metal. In such case, the enclosed outer ring 305 has a fixed shape and a fixed length.

The first adjustable structure may be attached to the enclosed outer ring 305, configured to adjust an internal wearing space IP of the wearable device 300_1 while a length of the enclosed outer ring 305 is fixed. In other words, a length of the enclosed outer ring 305 is a first length when the internal wearing space IP of the wearable device 300_1 is a first value, but the length of the enclosed outer ring 305 is still the first length when the internal wearing space IP of the wearable device 300_1 is adjusted from a first value to a second value.

The first adjustable structure may be various kinds of structures. In one embodiment, the first adjustable structure is an inflatable cuff. When the inflatable cuff is inflated, the internal wearing space IP becomes smaller. On the opposite, when the inflatable cuff is not inflated, the internal wearing space IP becomes larger. Please note, the structure of the ring is not limited to the example illustrated in FIG. 3B. Any ring which can change the internal wearing space thereof should fall in the scope of the present invention.

As above-mentioned, the wearable device 100 is in a loose state in FIG. 1 and is in a tight state in FIG. 2. Accordingly, in FIG. 1, the light emitted from the first light source LS_1 and the second light source LS_2 covers a larger portion of a blood vessel VE below the skin SK of the user and a smaller portion of the body tissue TI below the blood vessel VE. Oppositely, in FIG. 2, the light emitted from the first light source LS_1 and the second light source LS_2 covers a smaller portion of a blood vessel VE and a larger portion of the body tissue TI.

The states of the wearable device 100 may affect the optical signal. If the optical signal is not calibrated, the accuracy of the blood oxygen measurement may be affected. Accordingly, in following embodiment, a calibration method of the blood oxygen measurement is provided. In one embodiment, the optical signal comprises a first signal component and a second signal component, and the processing circuit 105 calibrates at least one of the first signal component and the second signal component according to the pressure to generate the calibrated optical signal. In one embodiment, the first signal component corresponds to pulsatile blood and the second signal component corresponds to non-pulsatile blood. Specifically, the pulsatile blood is blood flowing in arteries and the non-pulsatile blood is the blood flowing in veins. In one embodiment, the optical signal further comprises a third signal component related corresponding to body tissues besides arteries and veins.

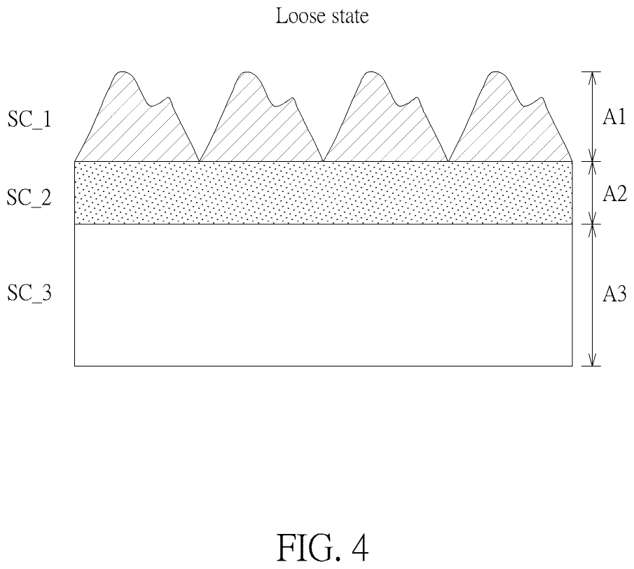

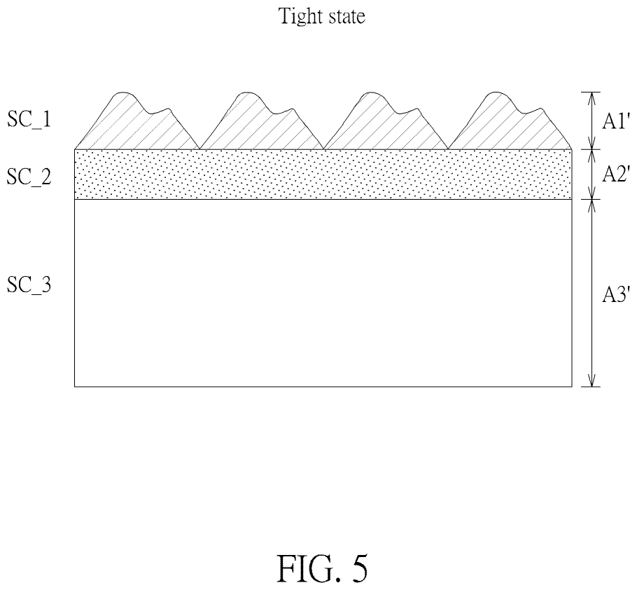

FIG. 4 and FIG. 5 are schematic diagrams illustrating the first signal component, the second signal component and the third signal component in different states of the wearable device, according to embodiments of the present invention. In the embodiments of FIG. 4 and FIG. 5, the first signal component SC_1, the second signal component SC_2 and the third signal component SC_3 respectively correspond to pulsatile blood, non-pulsatile blood and body tissues. Further, the embodiment of FIG. 4 corresponds to the above-mentioned loose state, and the embodiment of FIG. 5 corresponds to the above-mentioned tight state.

As above-mentioned, the light emitted from the first light source LS_1 and the second light source LS_2 covers a larger portion of the blood vessel VE and a smaller portion of the body tissue TI in FIG. 1 (the loose state). Oppositely, the light emitted from the first light source LS_1 and the second light source LS_2 covers a smaller portion of a blood vessel VE and a larger portion of the body tissue TI (the tight state). Accordingly, the amplitudes of the first signal component SC_1 and the second signal component SC_2, which correspond to the pulsatile blood and the non-pulsatile blood, will decrease. Thus, the amplitude A1′ is smaller than the amplitude A1, and the amplitude A2′ is smaller than the amplitude A2. Oppositely, the amplitude A3′ is larger than the amplitude A3.

In such case, the ratio between at least two of amplitudes A1, A2, A3 and the relations between at least two of amplitudes A1′, A2′, A3′ may be different. For example, the ratio between the amplitudes A1 and A2 is different from the ratio between the amplitudes A1′ and A2′. For another example, the ratio between the amplitudes A1 and A3 is different from the ratio between the amplitudes A1′ and A3′. As above-mentioned, the conditions of the amplitudes of the optical signals change in different states. Therefore, if the blood oxygen measurement is not calibrated corresponding to the condition variation of the optical signals, the blood oxygen measurement may be non-accurate.

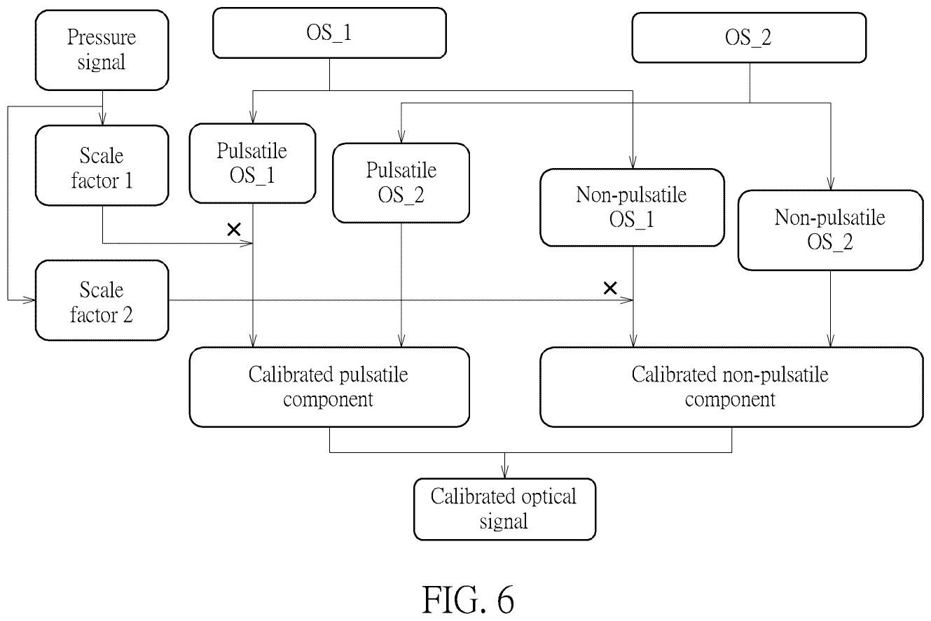

As above-mentioned, the pressure sensed by the pressure sensor 103 may correspond to the state of the wearable device 100, and the optical signal vary corresponding to the state of the wearable device 100, thus the optical signal may be calibrated according to the pressure sensed by the pressure sensor. FIG. 6 is a flow chart illustrating a calibration method for the first signal component or the second signal component, according to embodiments of the present invention. In the embodiment of FIG. 6, the optical signal OS_1 means the optical signal generated based on the light emitted from the first light source LS_1, and the optical signal OS_2 means the optical signal generated based on the light emitted from the second light source LS_2. The optical signal OS_1 comprises the first signal component (pulsatile OS_1) and the second signal component (non-pulsatile OS_1). Similarly, the optical signal OS_2 comprises the first signal component (pulsatile OS_2) and the second signal component (non-pulsatile OS_2). In one embodiment, the non-pulsatile signals mean DC (Direct Current) signals which may not be affected by blood. On the opposite, the pulsatile signals mean AC (Alternating Current) signals which may be affected by blood. Accordingly, different scale factors can be provided to different types of signals based on the differences of DC signals and AC signals. The steps of providing different scale factors will be explained for more detail in following descriptions.

In the embodiment of FIG. 6, the pulsatile OS_1 and the non-pulsatile OS_1 are respectively scaled up by a scale factor 1 and a scale factor 2, if the pressure is over a first pressure threshold (i.e., in the tight state). Such step may be used to scale up the pulsatile OS_2 or the non-pulsatile OS_2. Accordingly, the embodiment of FIG. 6 can be regarded as: scales up at least portion of the first signal component or at least portion of the second signal component if the pressure is over a first pressure threshold. As stated above, signal differences are caused by different light covering portions (e.g., thickness) of tissues, arteries or veins in the tight state and the loose state. Thus, the scale factors may be provided based on these signal differences.

The calibration of the optical signal is not limited to the steps illustrated in FIG. 6. In one embodiment, the third signal component SC_3 is scaled down if the pressure is over the first pressure threshold. In another embodiment, at least portion of the first signal component SC_1 or at least portion of the second signal component SC_2 is scaled down if the pressure is below a second pressure threshold (i.e., in the loose state). The second pressure threshold is smaller than or equal to the first pressure threshold. In such case, the third signal component SC_3 may be scaled up if the pressure is below the second pressure threshold. Additionally, in one embodiment, a ratio between the second signal component SC_2 and the third signal component SC_3 is changed by the processing circuit 105 according to the pressure.

In above-mentioned embodiments, the second signal component SC_2 and the third signal component SC_3 can be clearly distinguished from each other. However, in some embodiments, the second signal component SC_2 and the third signal component SC_3 are mixed to a fourth signal component and cannot be clearly distinguished from each other. In such case, amplitudes A2, A3 in FIG. 4 are integrated to an amplitude A4 and amplitudes A2′, A3′ in FIG. 5 are integrates to an amplitude A4′. Further, the ratio between the amplitudes A1, A4 is different from the ratio between the amplitudes A1′, A4′.

Besides, in the embodiment that the second signal component SC_2 and the third signal component SC_3 are mixed to a fourth signal component, the embodiment shown in FIG. 6 may be changed as following descriptions. In the embodiment of FIG. 6, the pulsatile OS_1 and the non-pulsatile OS_1 are respectively scaled up by a scale factor 1 and a scale factor 2, if the pressure is over a first pressure threshold (i.e., in the tight state). Such step may be used to scale up the pulsatile OS_2 or the non-pulsatile OS_2. Accordingly, the embodiment of FIG. 6 can be regarded as: scales up at least portion of the first signal component or at least portion of the fourth signal component if the pressure is over a first pressure threshold. Please note such step can also be regarded as: scales up at least portion of the first signal component or at least portion of the second signal component if the pressure is over a first pressure threshold, since the second signal component is contained in the fourth signal component. As stated above, signal differences are caused by different light covering portions (e.g., thickness) of tissues, arteries or veins in the tight state and the loose state. Thus, the scale factors may be provided based on these signal differences.

In one embodiment, at least portion of the first signal component SC_1 or at least portion of the fourth signal component is scaled down if the pressure is below a second pressure threshold (i.e., in the loose state). The second pressure threshold is smaller than or equal to the first pressure threshold. Similarly, such step can be regarded as: at least portion of the first signal component SC_1 or at least portion of the second signal component is scaled down if the pressure is below a second pressure threshold, since the second signal component is contained in the fourth signal component. Additionally, in one embodiment, a ratio between the second signal component SC_2 and the third signal component SC_3 is changed by the processing circuit 105 according to the pressure, since a scale factor is provided to process the fourth signal component.

Various methods can be used to implement the calibration steps. In one embodiment, a table which contains the relations between scaling factors and the pressure may be pre-generated and stored in the wearable device. The processing circuit 105 may perform the calibration steps according to the pressure and the table.

It will be appreciated the optical signal is not limited to comprise three signal components, and the types of the signal components are not limited to the above-mentioned examples. Also, the calibration steps are not limited to the above-mentioned examples and may vary corresponding to different signal components.

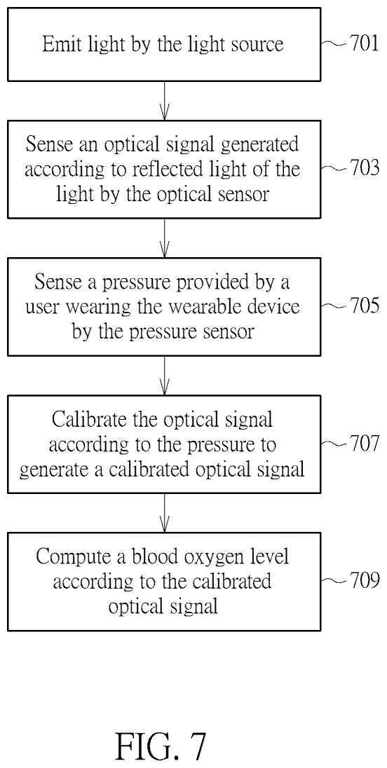

In view of above-mentioned embodiments, a blood oxygen measurement method is provided, which can be applied to a wearable device with at least one light source, an optical sensor and a pressure sensor. The wearable device further comprises a pressure adjusting structure configured to adjust an internal wearing space of the wearable device, wherein the pressure changes corresponding to the internal wearing space. FIG. 7 is a flow chart illustrating a blood oxygen measurement method according to one embodiment of the present invention, which comprises following steps:

### Step 701

Emit light by the light source (e.g., the first light source LS_1 and the second light source LS_1).

### Step 703

Sense an optical signal generated according to reflected light of the light by the optical sensor (e.g., the optical sensor 101).

### Step 705

Sense a pressure provided by a user wearing the wearable device by the pressure sensor (e.g., the pressure sensor 103).

### Step 707

Calibrate the optical signal according to the pressure to generate a calibrated optical signal (e.g., by the processing circuit 105).

### Step 709

Compute a blood oxygen level according to the calibrated optical signal.

Other detail steps of the blood oxygen measurement method may be acquired based on above-mentioned embodiments. Descriptions thereof are omitted for brevity here.

In view of above-mentioned embodiments, the optical signal may be calibrated corresponding to the state of the wearable device. By this way, the blood oxygen measurement may be more accurate.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A wearable device with a blood oxygen measurement function, comprising:
at least one light source, configured to emit light;
an optical sensor, configured to sense an optical signal generated according to reflected light of the light;
a pressure sensor, configured to sense a pressure provided by a user wearing the wearable device;
a processing circuit, configured to calibrate the optical signal to generate a calibrated optical signal, and configured to compute a blood oxygen level according to the calibrated optical signal; and
a pressure adjusting structure, configured to adjust an internal wearing space of the wearable device, wherein the pressure changes corresponding to the internal wearing space.

2. The wearable device of claim 1,
wherein the optical signal comprises a first signal component and a second signal component;
wherein the processing circuit calibrates at least one of the first signal component and the second signal component according to the pressure to generate the calibrated optical signal.

3. The wearable device of claim 2, wherein the first signal component corresponds to pulsatile blood and the second signal component corresponds to non-pulsatile blood.

4. The wearable device of claim 3, wherein the pulsatile blood is blood flowing in arteries and the non-pulsatile blood is the blood flowing in veins.

5. The wearable device of claim 2, wherein the processing circuit scales up at least portion of the first signal component or at least portion the second signal component if the pressure is over a first pressure threshold.

6. The wearable device of claim 5, wherein the optical signal further comprises a third signal component related corresponding to tissues besides arteries and veins, wherein the processing circuit scales down the third signal component if the pressure is over the first pressure threshold.

7. The wearable device of claim 2, wherein the processing circuit scales down at least portion of the first signal component or at least portion of the second signal component if the pressure is below a second pressure threshold.

8. The wearable device of claim 7, wherein the optical signal further comprises a third signal component related corresponding to tissues besides arteries and veins, wherein the processing circuit scales up the third signal component if the pressure is below the second pressure threshold.

9. The wearable device of claim 2,
wherein the first signal component corresponds to pulsatile blood flowing in arteries, the second signal component corresponds to non-pulsatile blood flowing in veins, and the optical signal further comprises a third signal component related corresponding to tissues besides arteries and veins;
wherein the processing circuit calibrates a ratio change between the second signal component and the third signal component according to the pressure.

10. The wearable device of claim 1, wherein the light source comprises at least two light sources which emit light with different light wave lengths.

11. A blood oxygen measurement method, applied to a wearable device with at least one light source, an optical sensor and a pressure sensor, comprising:
(a) emitting light by the light source;
(b) sensing an optical signal generated according to reflected light of the light by the optical sensor;
(c) sensing a pressure provided by a user wearing the wearable device by the pressure sensor;
(d) calibrating the optical signal according to the pressure to generate a calibrated optical signal; and
(e) computing a blood oxygen level according to the calibrated optical signal;
wherein the wearable device further comprises a pressure adjusting structure configured to adjust an internal wearing space of the wearable device, wherein the pressure changes corresponding to the internal wearing space.

12. The blood oxygen measurement method of claim 11,
wherein the optical signal comprises a first signal component and a second signal component;
wherein the step (d) calibrates at least one of the first signal component and the second signal component according to the pressure to generate the calibrated optical signal.

13. The blood oxygen measurement method of claim 12, wherein the first signal component corresponds to pulsatile blood and the second signal component corresponds to non-pulsatile blood.

14. The blood oxygen measurement method of claim 13, wherein the pulsatile blood is blood flowing in arteries and the non-pulsatile blood is the blood flowing in veins.

15. The blood oxygen measurement method of claim 12, wherein the step (d) scales up at least one of the first signal component and the second signal component if the pressure is over a first pressure threshold.

16. The blood oxygen measurement method of claim 15, wherein the optical signal further comprises a third signal component related corresponding to tissues besides arteries and veins, wherein the step (d) scales down the third signal component if the pressure is over the first pressure threshold.

17. The blood oxygen measurement method of claim 12, wherein the step (d) scales down at least one of the first signal component and the second signal component if the pressure is below a second pressure threshold.

18. The blood oxygen measurement method of claim 17, wherein the optical signal further comprises a third signal component related corresponding to tissues besides arteries and veins, wherein the step (d) scales up the third signal component if the pressure is below the second pressure threshold.

19. The blood oxygen measurement method of claim 12,
wherein the first signal component corresponds to pulsatile blood flowing in arteries, the second signal component corresponds to non-pulsatile blood flowing in veins, and the optical signal further comprises a third signal component related corresponding to tissues besides arteries and veins;
wherein the step (d) calibrates a ratio change between the second signal component and the third signal component according to the pressure.

20. The blood oxygen measurement method of claim 12, wherein the light source comprises at least two light sources which emit light with different light wave lengths.
